(19) United States
(12) Patent Application Publication
(10) Pub. No.: US 2024/0315708 A1
(43) Pub. Date: Sep. 26, 2024

(54) TOOL AND METHOD FOR IMPLANTING FUSION DEVICE INTO SACROILIAC JOINT

(71) Applicant: VGI Medical, LLC, Largo, FL (US)

(72) Inventor: Tov Inge Vestgaarden, Madeira Beach, FL (US)

(21) Appl. No.: 18/515,470

(22) Filed: Nov. 21, 2023

Related U.S. Application Data
(63) Continuation of application No. 15/889,587, filed on Feb. 6, 2018, now Pat. No. 11,819,226.
Application No. 15/889,587 is a continuation of application No. 13/790,416, filed on Mar. 8, 2013, now Pat. No. 9,883,874.

(51) Int. Cl. A61B 17/17; A61B 17/70; A61B 17/88; A61B 17/90; A61F 2/46

(52) U.S. Cl. CPC A61B 17/1739; A61B 17/17; A61B 17/1757; A61B 17/8894; A61F 2/4603; A61F 2/4611; A61B 17/7055; A61B 17/90

(57) ABSTRACT

Tools for positioning an implant into the sacroiliac joint. A directional cannula includes a main body having a bore that receives the implant. A cut-out, allowing access to the joint, is formed in a leading end of the main body. A pair of parallel prongs extend from the leading end of the main body in transversely spaced apart relation to one another. A drill guide has a main body of rectangular transverse cross-section and a cylindrical head formed integrally with the main body. A transverse width-reducing step is formed in the main body near its distal end. First and second bores are formed in the cylindrical head and in the main body. Both bores are eccentric relative to the drill guide longitudinal axis of symmetry. The drill guide is rotated 180° after first and second drilling operations, prior to third and fourth drilling operations.

### BACKGROUND OF THE INVENTION

### 1. Field of the Invention

This invention relates, generally, to surgical instruments and methods. More particularly, it relates to a tool for introducing a fusion device, also known as an implant or fusion implant, into a sacroiliac joint.

### 2. Description of the Prior Art

Placing a fusion implant into the sacroiliac area is difficult because the ilium protrudes and blocks easy access to the site. Part of the ilium can be cut and removed to improve access, but such cutting weakens the ilium, extends the time required for surgery and recovery from surgery, and increases the patient's pain.

New tools are needed that would eliminate the need to cut the ilium.

However, in view of the art considered as a whole at the time the present invention was made, it was not obvious to those of ordinary skill in the art how the needed tools could be provided.

### SUMMARY OF THE INVENTION

The long-standing but heretofore unfulfilled need for a device that facilitates the insertion of an implant into the sacroiliac joint is now met by a new, useful, and non-obvious invention.

The inventive structure includes a directional cannula having a main body of elongate cylindrical extent. A bore having the shape of a spinal fusion implant is formed in the main body. That bore is also configured to receive a drill guide therein, if needed.

A cut-out is formed in a leading end of the main body by a radial cut, i.e., a cut that is normal to a longitudinal axis of said main body. The radial cut extends less than half-way through the main body. The cut-out is also formed by a second longitudinally-extending cut that extends from a leading end of the main body to the radial cut at a point of deepest penetration of the radial cut.

The implant is captured within the bore even when the implant travels to the cut-out or notch formed in the main body and is exposed to view. Since the notch has a radial depth less than the diameter of the bore, the implant is more than half-surrounded by the lumen and cannot fall therefrom.

A pair of parallel prongs extends longitudinally from the leading end of the main body in transversely spaced apart relation to one another. The prongs are adapted to enter the sacroiliac joint, which is the space between the ilium and sacrum.

The novel drill guide includes an elongate main body having a rectangular transverse cross-section, an enlarged cylindrical head formed integrally with the main body at a proximal end of the main body, and a transverse width-reducing step formed in the main body near a distal end of the main body.

A first longitudinally-extending bore is formed in the enlarged cylindrical head and in the main body. The first bore is eccentric relative to a longitudinal axis of symmetry of the drill guide.

A second longitudinally-extending bore is also formed in the enlarged cylindrical head and in the main body. The second bore is also eccentric relative to the longitudinal axis of symmetry of the drill guide and the second bore is parallel to the first bore.

A drill bit is sequentially placed within the first and second bores to create clearance space in the ilium when the drill guide is received within the bore of the directional cannula. The drill guide is then removed from the bore of the directional cannula, rotated one hundred eighty degrees (180°), and the drill bit is again sequentially placed within the first and second bores to create clearance space in the sacrum.

An important object of this invention is to facilitate the implanting of a fusion implant in the sacroiliac area.

A more specific object is to accomplish the first object in the absence of removing substantial parts of the ilium.

Another important object is to provide tools that produce consistent results from patient to patient.

These and other important objects, advantages, and features of the invention will become clear as this disclosure proceeds.

The invention accordingly comprises the features of construction, combination of elements, and arrangement of parts that will be exemplified in the disclosure set forth hereinafter and the scope of the invention will be indicated in the claims.

### BRIEF DESCRIPTION OF THE DRAWINGS

For a fuller understanding of the nature and objects of the invention, reference should be made to the following detailed disclosure, taken in connection with the accompanying drawings, in which:

FIG. 1A is an X-ray view depicting the first guide wire placed on the skin to identify the location of the sacroiliac joint;

FIG. 1B is a diagrammatic view of a mark made on a patient's skin to indicate the position of the guide wire used to locate the sacroiliac joint in FIG. 1A;

FIG. 2A is an X-ray view depicting the second guide wire placed on the skin to identify the superior portion of the sacroiliac joint;

FIG. 2B is a diagrammatic view of a mark made on a patient's skin to indicate the position of the guide wire used to identify the superior portion of the sacroiliac joint in FIG. 2A;

FIG. 3A is an X-ray view depicting the third guide wire placed on the skin to identify the inferior portion of the sacroiliac joint;

FIG. 3B is a diagrammatic view of a mark made on a patient's skin to indicate the position of the guide wire used to identify the inferior portion of the sacroiliac joint in FIG. 3A;

FIG. 4 is a diagrammatic view of three incision points that are marked on the patient's skin after the markings in FIGS. 1B, 2B, and 3B have been made;

FIG. 5 is an X-ray view depicting a guide wire inserted through the central incision point of the three incision points of FIG. 4;

FIG. 6A is a perspective view of a joint locator;

FIG. 6B is a side elevational view of the joint locator;

FIG. 6C is a top plan view of the joint locator;

FIG. 7A is a perspective view of a novel directional cannula;

FIG. 7B is a top plan view of the directional cannula of FIG. 7A;

FIG. 7C is a side elevational view of the directional cannula of FIG. 7A;

FIG. 7D is a bottom plan view of the directional cannula of FIG. 7A;

FIG. 7E is an end view of the leading end of the directional cannula of FIG. 7A;

FIG. 7F is an end view of the trailing end of the directional cannula of FIG. 7A;

FIG. 7G is a side elevational view similar to FIG. 7C, but depicting an implant exposed to view by a notch but captured within a lumen so that it cannot fall;

FIG. 7H is an end view taken along lines 7H-7H in FIG. 7G;

FIG. 8A is a perspective view of a novel drill guide;

FIG. 8B is a side elevational view of the drill guide of FIG. 8A;

FIG. 8C is an end elevational view of the leading end of the drill guide of FIG. 8A;

FIG. 8D is an end elevational view of the trailing end of the drill guide of FIG. 8A;

FIG. 9 is a perspective view of a drill bit;

FIG. 10 is a perspective view of a fusion implant device;

FIG. 11 is a perspective view of a tamp;

FIG. 12A is a front elevational diagrammatic representation of a sacrum flanked by a pair of iliums, showing two (2) sacroiliac joints;

FIG. 12B is a first perspective view of the diagrammatic representation of FIG. 12A, indicating how the iliums protrude forwardly relative to the sacrum;

FIG. 12C is a second perspective view of the diagrammatic representation of FIG. 12A;

FIG. 12D is a view like FIG. 12C but adding a notched cannula, showing how the notch accommodates a protruding ilium;

FIG. 12E depicts the same parts as FIG. 12D but taken from a left perspective;

FIG. 12F is the same view as FIG. 12E but including the drill bit;

FIG. 12G is the same as FIG. 12F but taken from a right perspective;

FIG. 12H depicts the cavity after drilling is complete and the cannula and drill bit have been withdrawn;

FIG. 12I is a like FIG. 12H but from a different perspective;

FIG. 12J depicts an implant in the drilled cavity; and

FIG. 12K depicts the same structure as FIG. 12J but from a different perspective.

### DETAILED DESCRIPTION OF THE PREFERRED EMBODIMENT

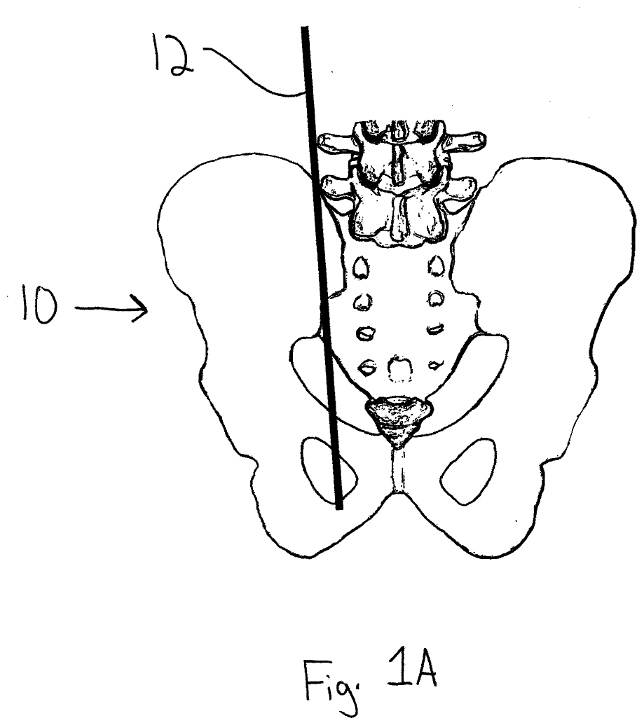

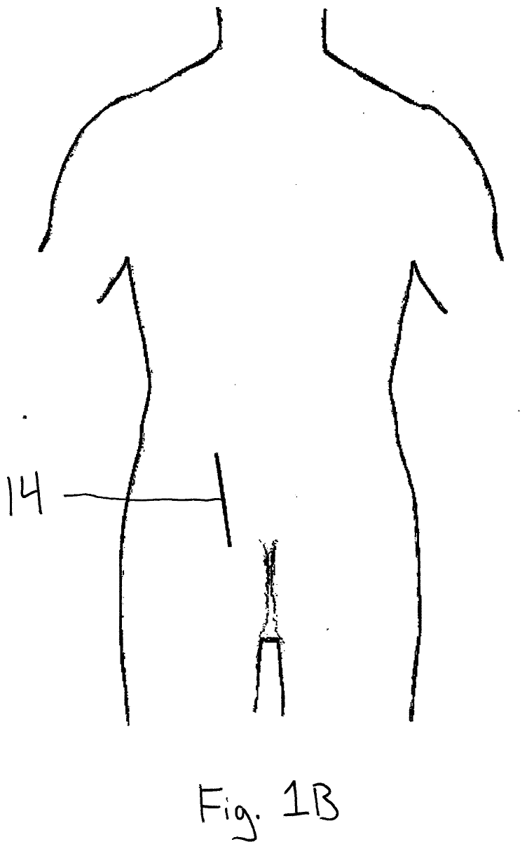

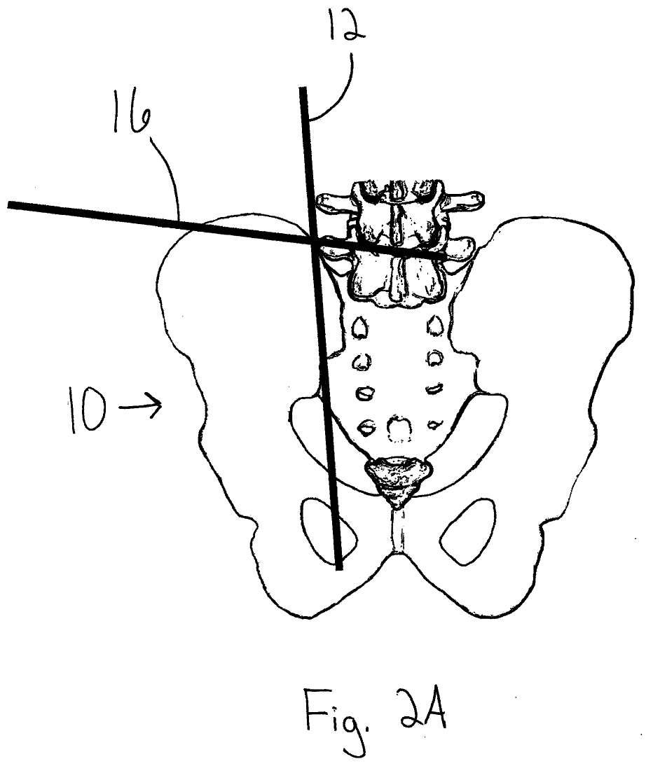

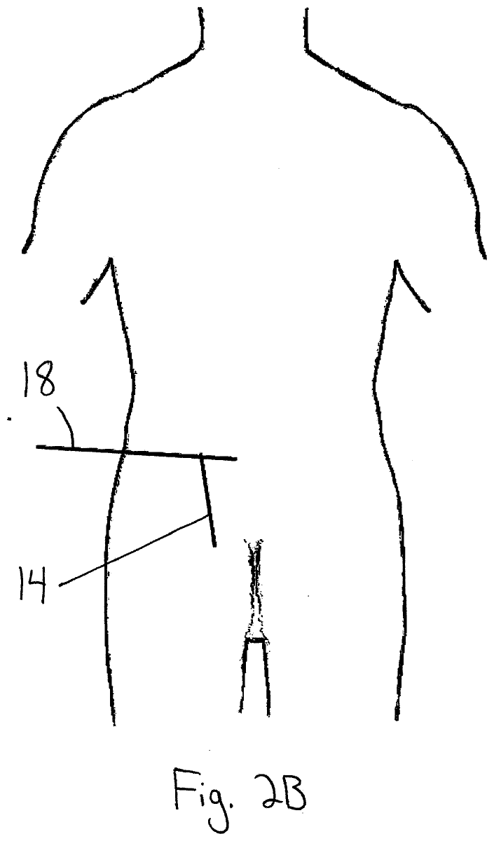

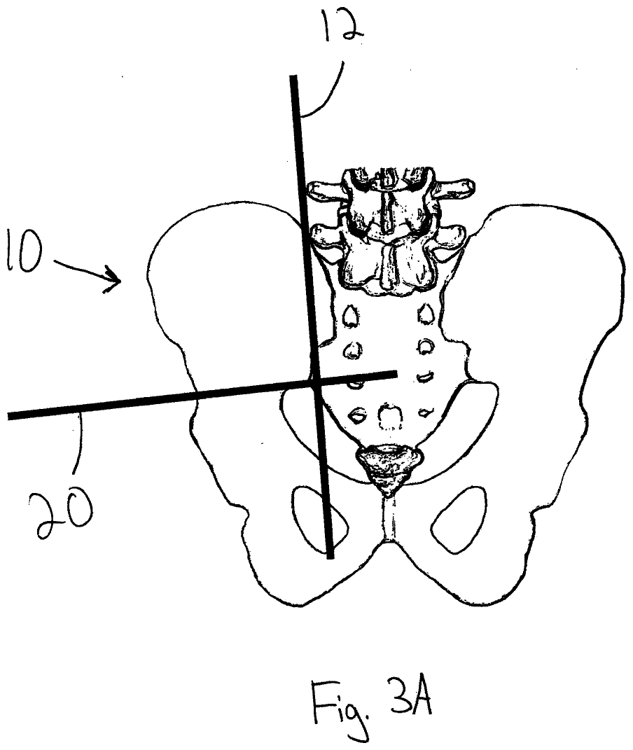

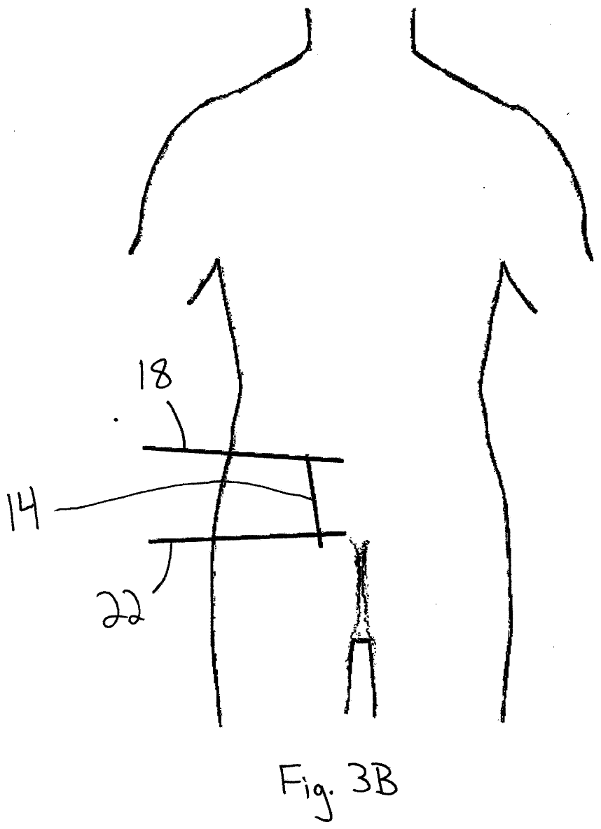

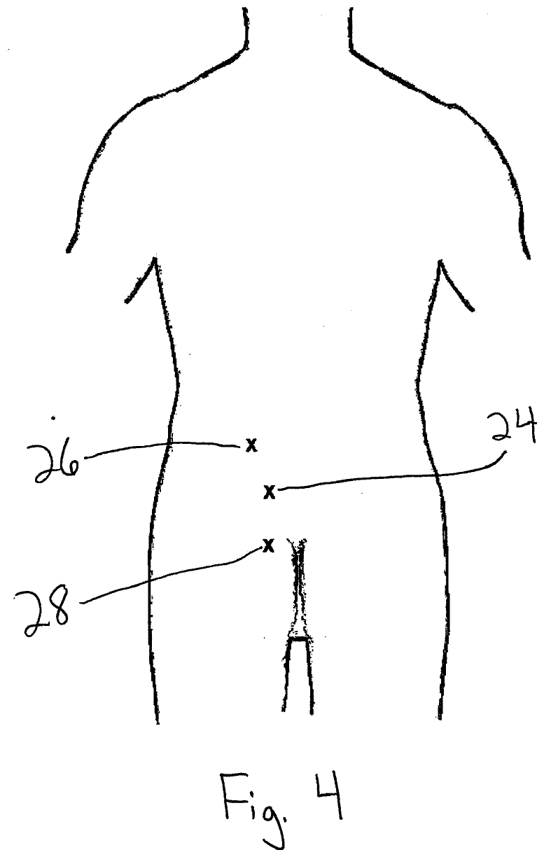

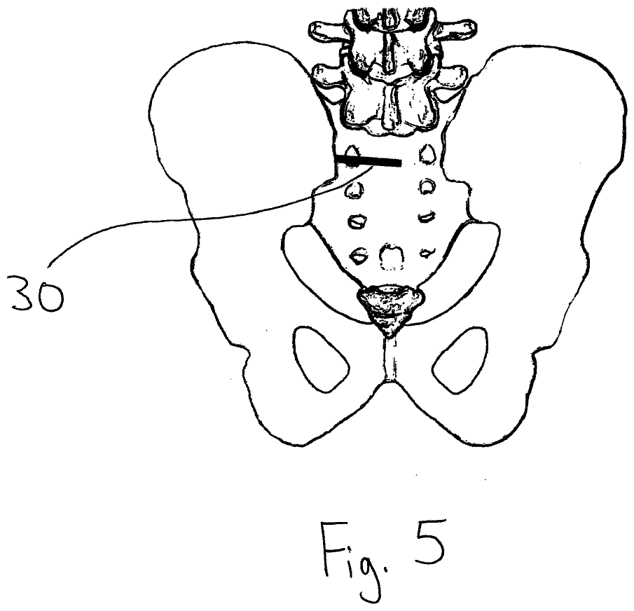

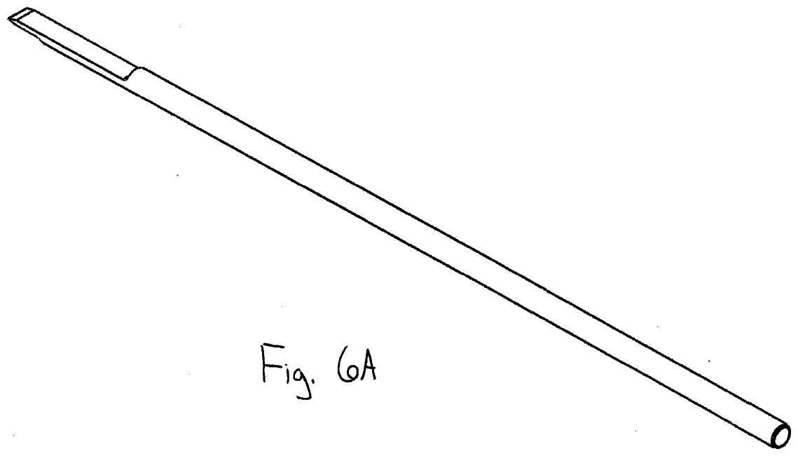

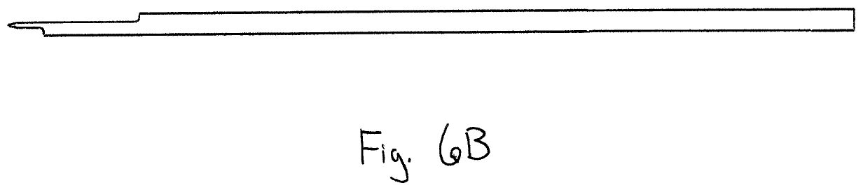

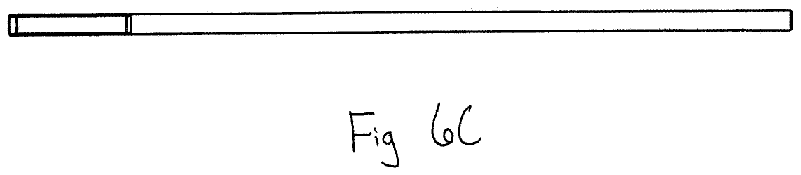

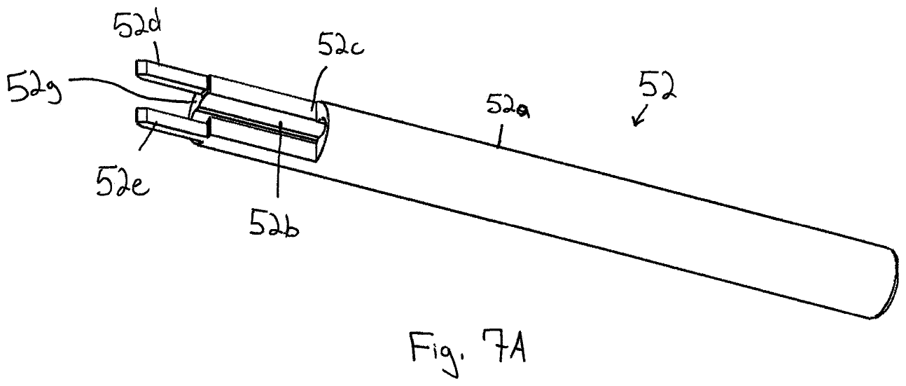

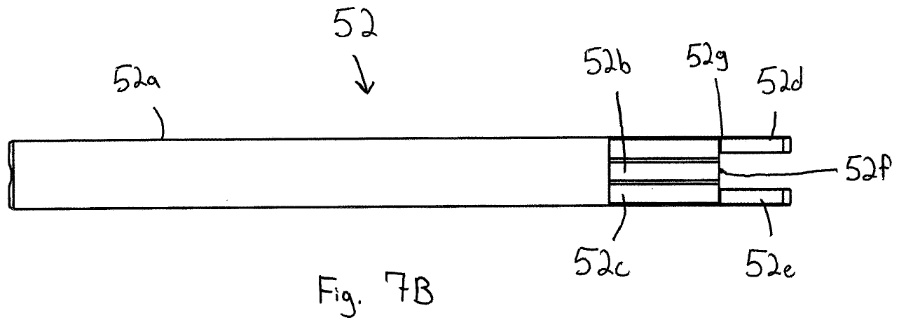

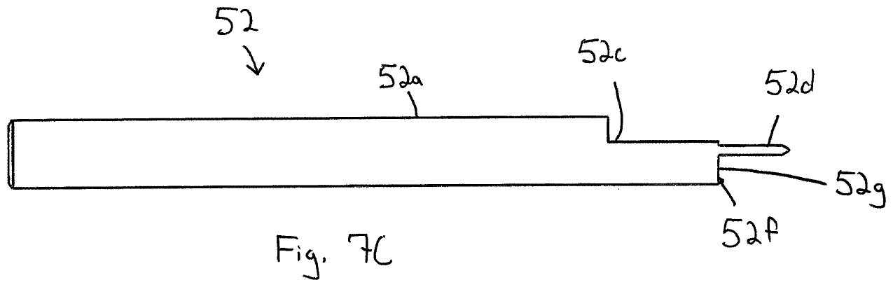

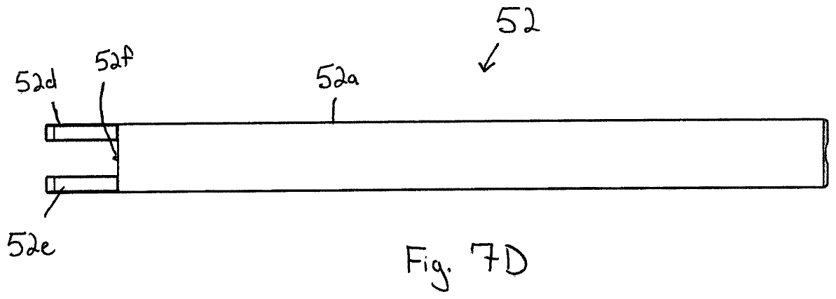

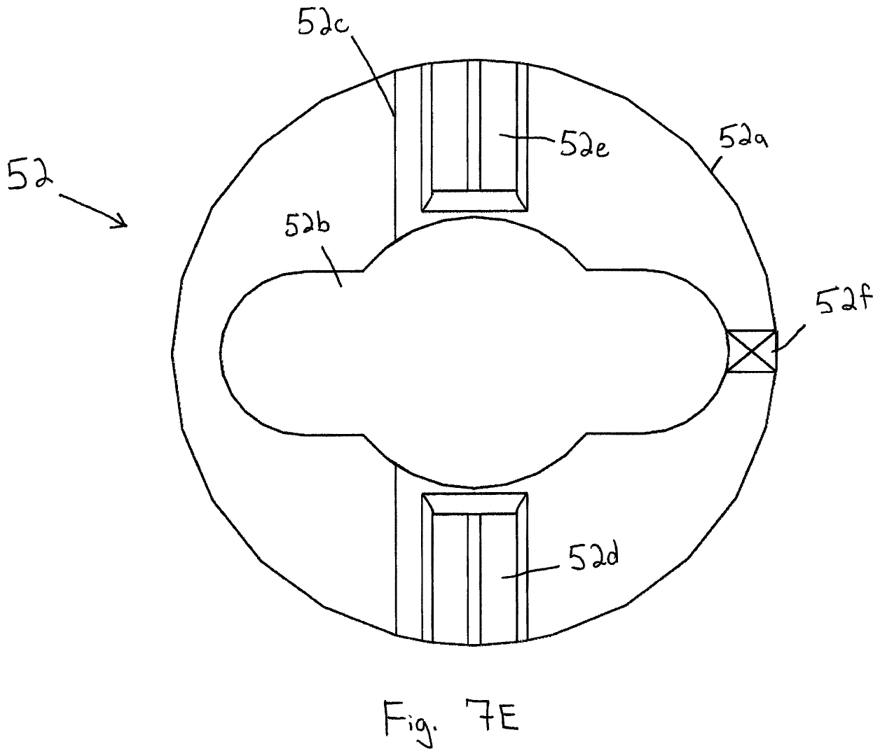

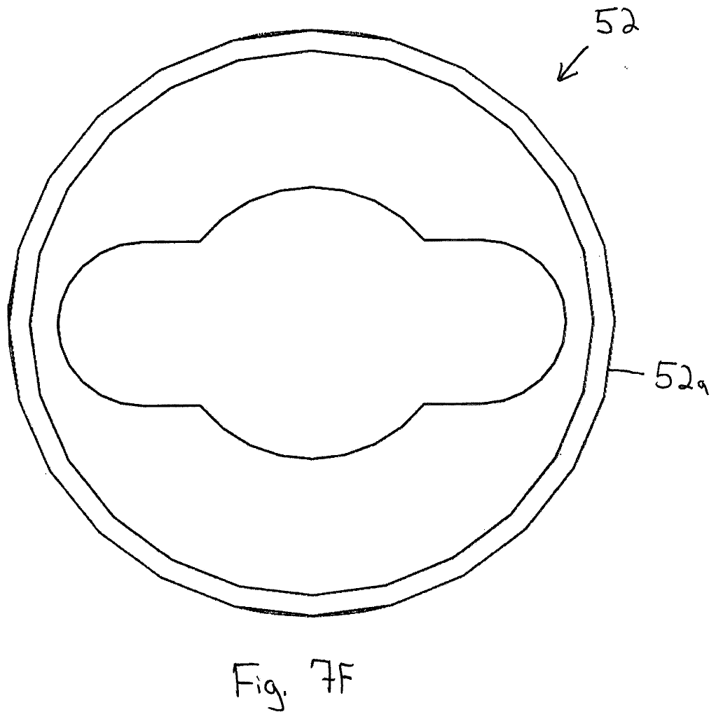

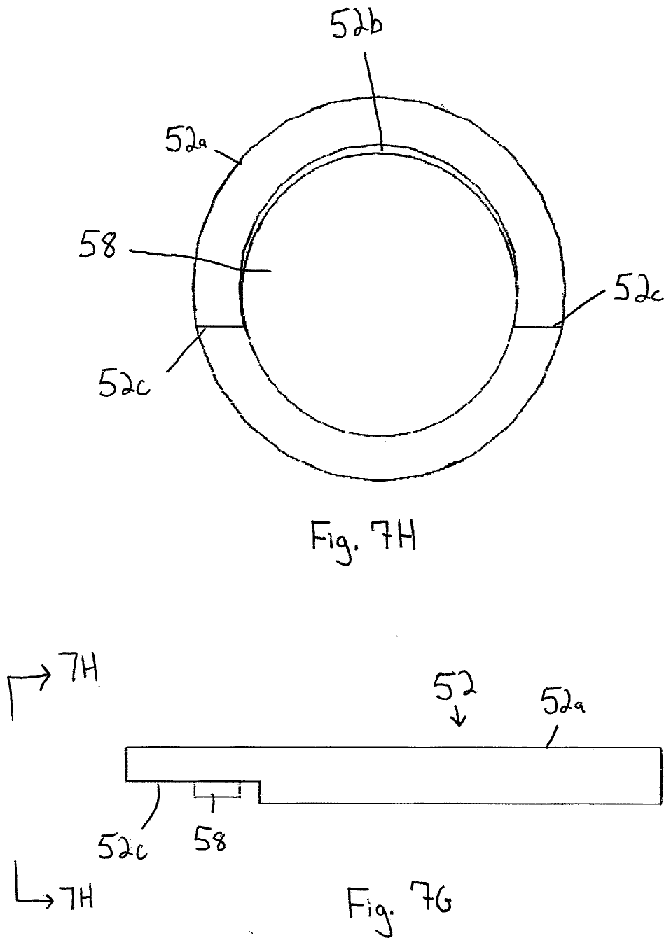

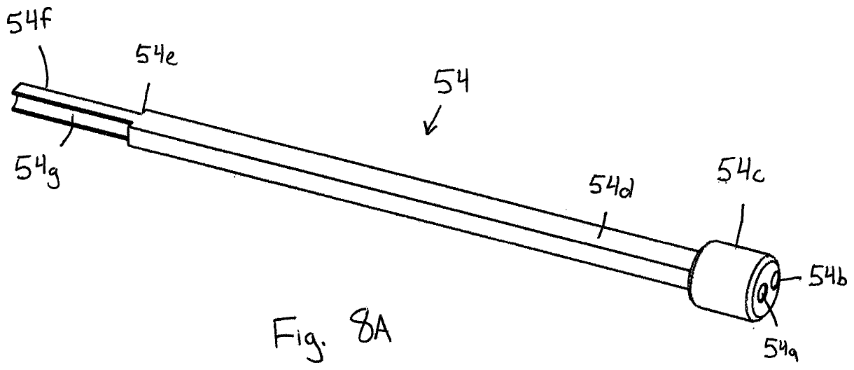

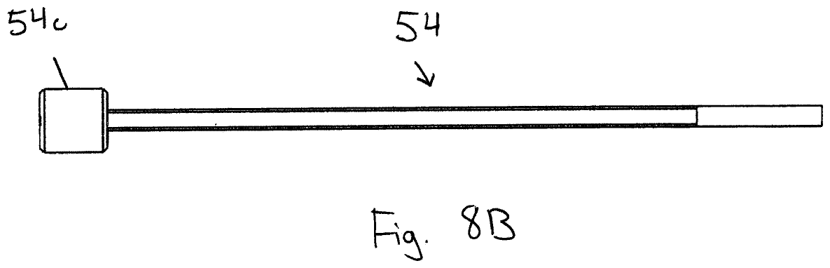

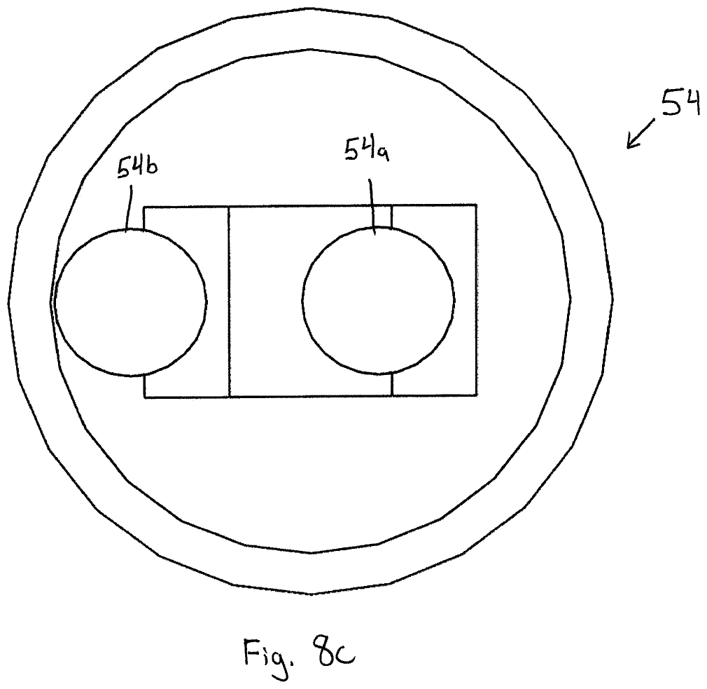

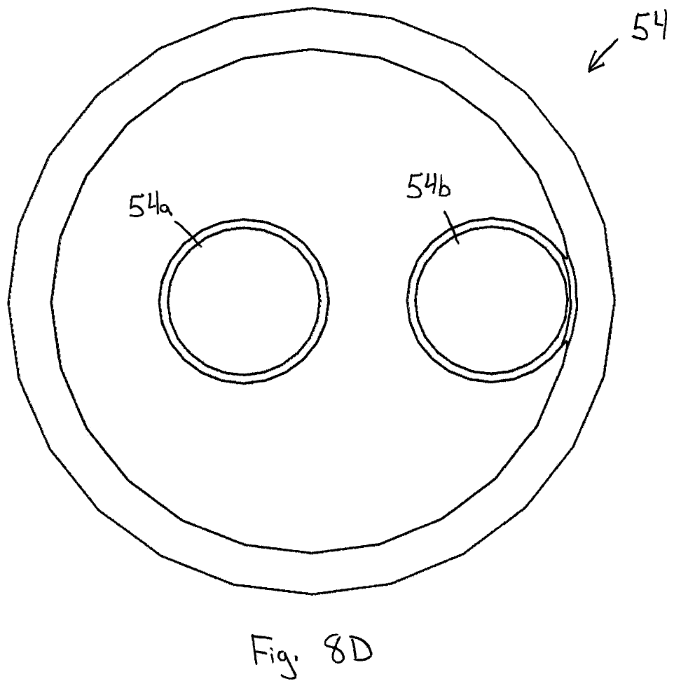

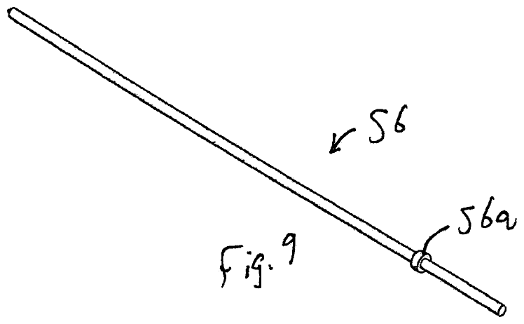

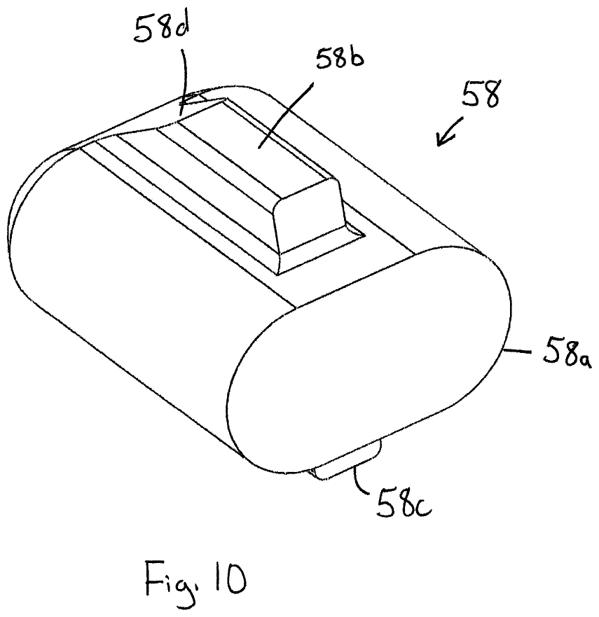

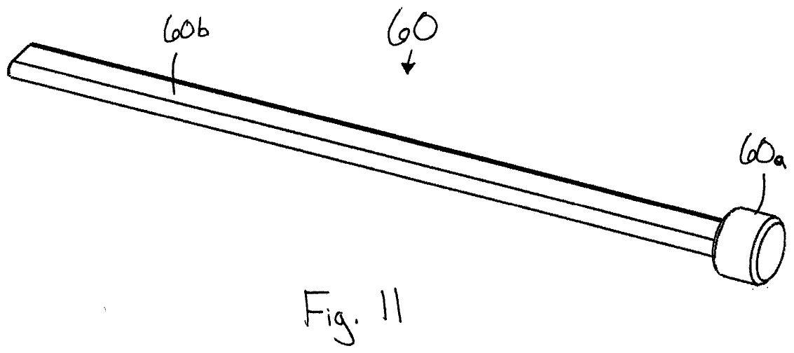

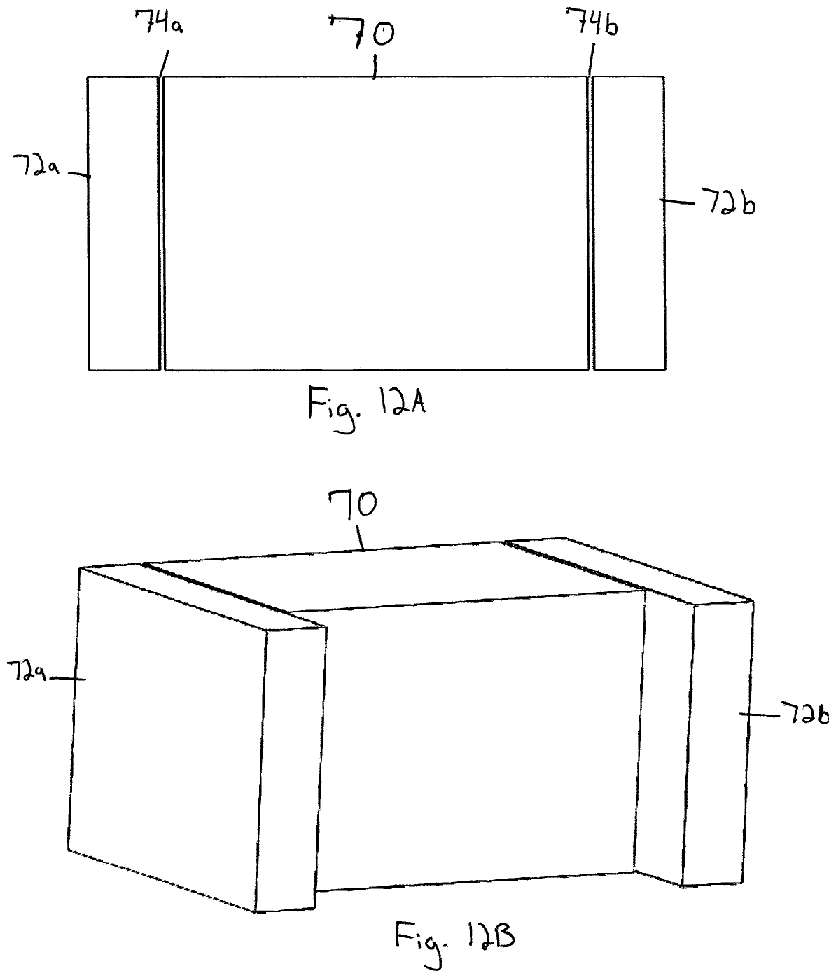

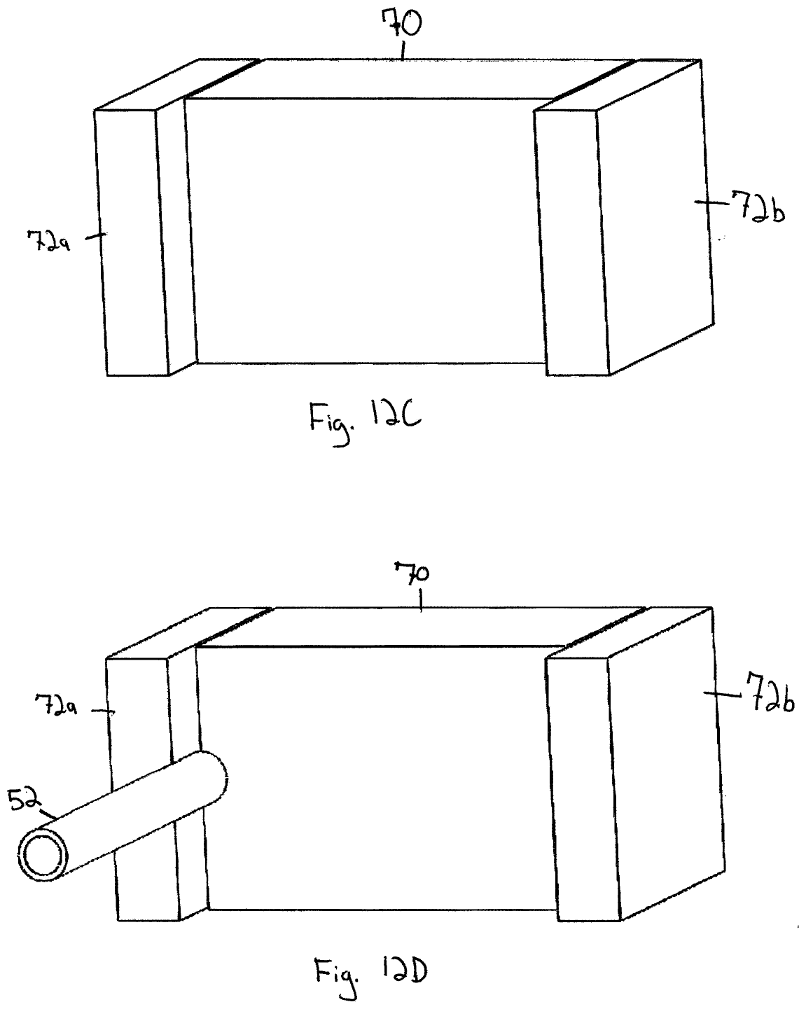

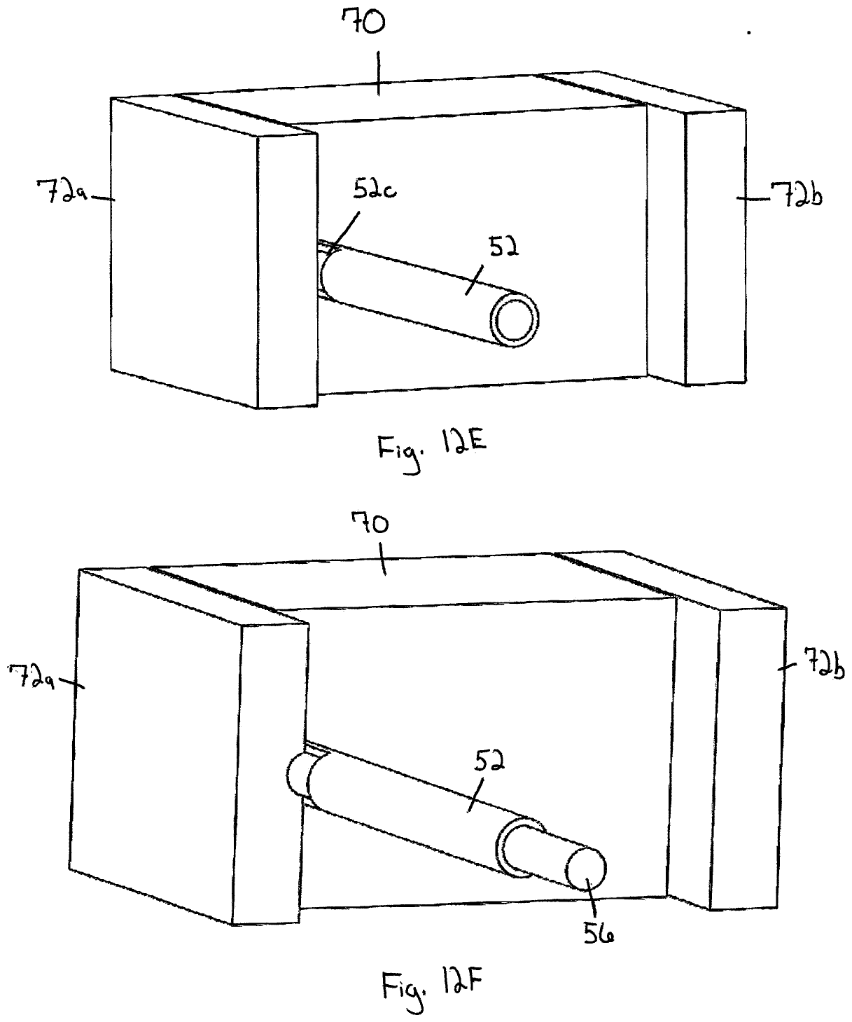

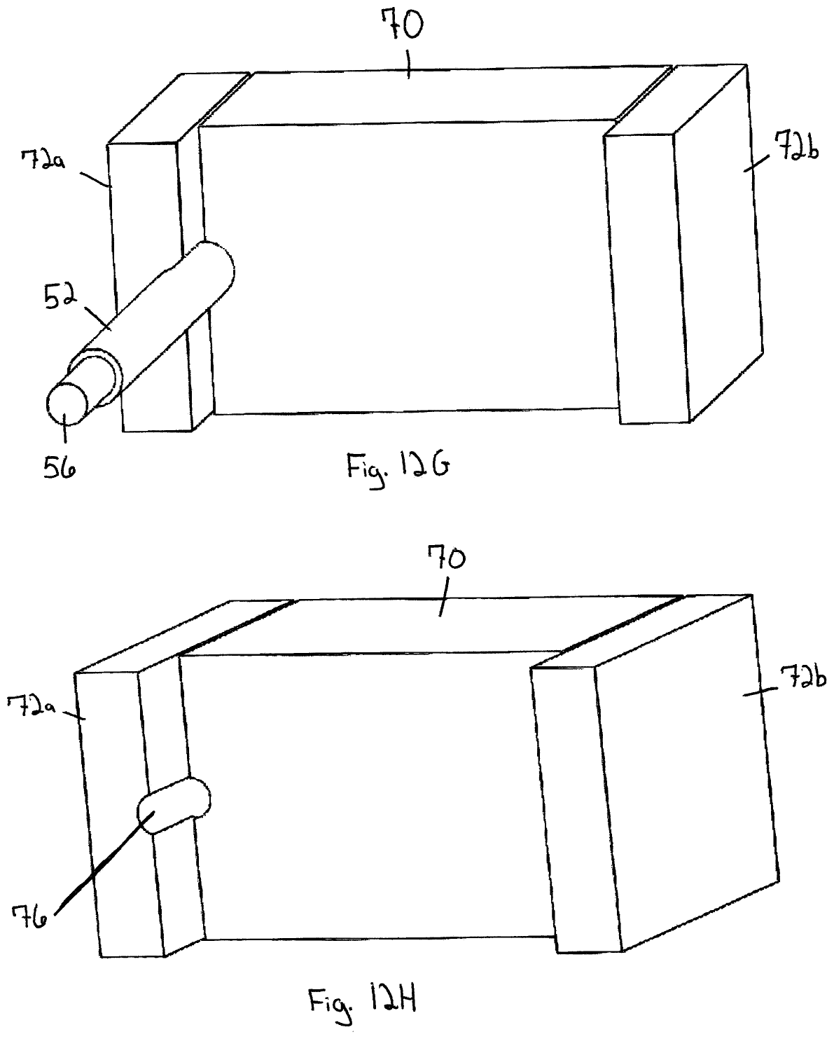

FIGS. 1A-8D depict an illustrative embodiment of the novel instrument and the novel method steps with which it is used. The sacroiliac (SI) joint of a patient is denoted as a whole by the reference numeral 10.

The novel method steps include the steps of taking anterior, posterior, and lateral X-ray views of the sacroiliac (SI) area to identify the anatomy that is causing pain in a patient.

An entry point is established to gain access to the SI joint with an oblique (approximately thirty five degree (35°) angle) and a Ferguson angle measurement of curvature which is approximately ten to fifteen degrees (10-15°).

As depicted in FIG. 1A, first guide wire 12 is placed on top of the SI joint and as depicted in FIG. 1B, the patient's skin is marked with a first straight line 14 to indicate the position of first guide wire 12. First straight line 14 is drawn through SI joint 10 from the superior position of the joint to the inferior position of the joint.

As depicted in FIG. 2A, a second guide wire 16 is then placed over the superior section of joint 10 in intersecting relation to first guide wire 12. As depicted in FIG. 2B, the patient's skin is marked with a second straight line 18 to indicate the position of second guide wire 16.

A third guide wire 20, as depicted in FIG. 3A, is then placed over the inferior section of the joint in intersecting relation to first guide wire 12. The patient's skin, as depicted in FIG. 3B, is marked with a third straight line 22 to indicate the position of third guide wire 20.

Three incision points are then marked on the skin, as depicted in FIG. 4.

Center incision point 24 is midway between the points where the second and third lines 18 and 22 cross first line 12 and is spaced in the medial direction about three to five millimeters (3-5 mm) from first line 12.

Superior incision point 26 is spaced in the medial direction about three to five millimeters (3-5 mm) from the intersection of first line 12 and second line 14.

Inferior incision point 28 is spaced in the medial direction about three to five millimeters (3-5 mm) from the intersection of first line 12 and third line 22.

As depicted in FIG. 5, a fourth guide wire 30 is inserted through center incision point 24, with a superior/inferior angle perpendicular to the patient. The medial/lateral angle is the same as the oblique angle on the C-arm, which is approximately thirty-five degrees (35°). Fourth guide wire 30 is guided into the SI joint and an incision is made when guide wire 30 is properly positioned.

Joint locator 50, depicted in FIGS. 6A-C, is then placed over guide wire 30, i.e., in ensleeving relation to said guide wire 30. A surface of joint locater 50 is etched black and that black-etched surface is positioned so that it faces the ilium.

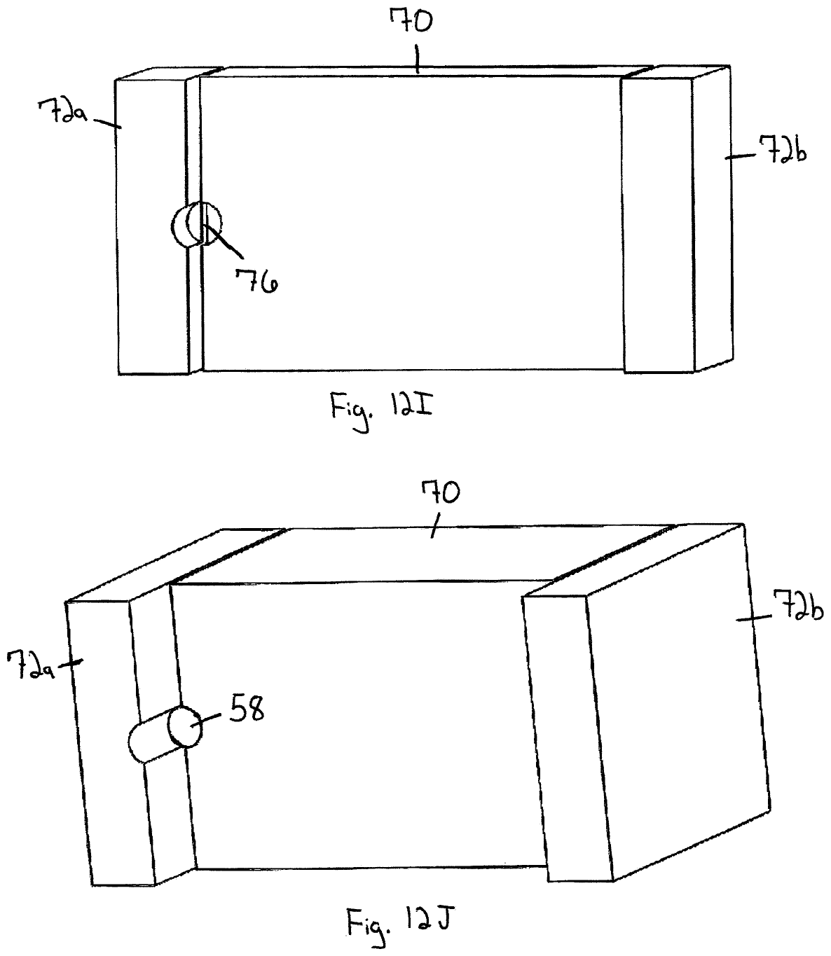

Directional cannula 52, depicted in FIGS. 7A-7G, is then placed over joint locator 50, i.e., in ensleeving relation to said joint locator. A surface of directional cannula 52 is etched black and that black-etched surface matches the black-etched surface of joint locator 50, i.e., the black-etched surface of directional cannula 52 is also positioned so that it faces the ilium.

Directional cannula 52 has a cylindrical main body 52a having longitudinally-extending central bore 52b formed therein. Central bore 52b has a transverse profile that matches the transverse profile of a fusion implant. A leading end of central bore 52b is exposed to view by cut-out or notch 52c. Two prongs 52d and 52e extend from said leading end in transversely spaced, parallel relation to one another. Prongs 52d and 52e enter the sacroiliac joint when directional cannula 52 is in use. Barb 52f is formed in the leading end of main body 52a as depicted in FIGS. 7B-7E. Barb 52f engages the sacrum to prevent slippage when prongs 52d and 52e enter into sacroiliac joint 10.

FIG. 7G depicts implant 58 that is captured within bore 52b. Implant 58 is exposed to view because it is in the region of bore 52b where notch 52c is formed. Since the radial depth of notch 52c is less than half the diameter of bore 52b, implant 58 cannot fall from bore/lumen 52b. In other words, as indicated in FIG. 7H, notch 52c creates a “C”-shaped bore where the two (2) spaced apart points of the “C” are closer together than the widest part of implant 58, thereby retaining the implant within bore 52b.

Joint locator 50 and guide wire 30 are then retracted, leaving directional cannula 52 in position. When so positioned, prongs 52d, 52e are disposed in sacroiliac joint 10. Barb 52f engages the sacrum to hold directional cannula 52 in position as aforesaid.

Drill guide 54, depicted in FIGS. 8A-8D, has longitudinally extending eccentric bores 54a, 54b formed therein. Head 54c is enlarged relative to main body 54d that is rectangular in transverse section. Step 54e is formed where the transverse extent of main body 54d is reduced. Drill bit-accommodating semi-circular grooves 54f and 54g are the continuation of bores 54a, 54b and are formed in opposite sides of the leading end of drill guide 54, said leading end being the part of main body 54d that extends distal of said step 54e.

Drill guide 54 is inserted into the central bore or lumen of directional cannula 52 towards the sacrum to verify placement of directional cannula 52 into the SI joint. The lumen of directional cannula 52 has a profile that enables it to slidingly receive drill guide 54 in the center of the larger implant-receiving lumen.

Drill bit 56, depicted in FIG. 9, having positive stop 56a is then inserted into eccentric bore 54a and said drill bit 56 is slid towards the sacrum until it abuts the sacrum.

The distance from the proximal end of head 54c of drill guide 54 to the lower side of positive stop 56a is then measured. Directional cannula 52 is properly seated in the SI joint if the measured distance is between twenty-five to thirty millimeters (25-30 mm). The proper seating can also be confirmed with a lateral X-ray view that shows the leading end of directional cannula 52 disposed flush with the sacrum.

Drill bit 56 is then inserted into eccentric drill guide bore 54a and a first cavity is created in the ilium by a first drilling, until positive stop 56a abuts the proximal end of directional cannula 52. Drill bit 56 is then withdrawn from bore 54a, placed into bore 54b, and a second cavity is formed in the ilium by a second drilling. The second drilling continues until drill bit 56 reaches positive stop 56a.

Drill guide 54 is then retracted from directional cannula 52 and rotated one hundred eighty degrees (180°). Drill bit 56 is then inserted into eccentric drill guide bore 54a and a first cavity is created in the sacrum by a third drilling that continues until drill bit 56 reaches positive stop 56a. Drill bit 56 is then withdrawn from bore 54a, placed into bore 54b, and a second cavity is formed in the sacrum by a fourth drilling. The fourth drilling continues until drill bit 56 reaches positive stop 56a. Drill bit 56 is then removed.

Due to the eccentricity of the bores and the rotation of the drill guide, all four cavities merge into a single cavity that accommodates the fusion implant.

When the drilling is completed, drill guide 54 is retracted from directional cannula 52 and fusion implant 58, depicted in FIG. 10, is inserted into the lumen of directional cannula 52. The chamfer is inserted downward.

Fusion implant 58 may take many forms and may be as simple as a dowel having a circular cross-section, i.e., the oval shape of main body 58a, upper and lower fins 58b, 58c, and the swept back leading edge 58d of said fins are not critical parts of the fusion implant.

Implant tamp 60, depicted in FIG. 11, includes head 60a and flat, elongate main body 60b. A positive stop, not numbered, is formed where main body 60b meets head 60a. Main body 60b is inserted into the lumen of directional cannula 52 to advance the implant. Head 60a is repeatedly tamped lightly with a hammer or other suitable tool, not illustrated, until the aforesaid positive stop abuts directional cannula 52. This should fully seat implant 58 in the SI joint, i.e., implant 58 should be countersunk into the SI joint by a distance of about three to five millimeters (3-5 mm). A lateral X-ray view is taken to confirm full deployment of implant 58.

As perhaps best understood in connection with FIGS. 7B and 7C, implant 58 emerges from bore 52b at cut-out or notch 52c. Notch 52c has an extent or depth less than half the diameter of cylindrical main body 52a as depicted in FIGS. 7C and 7E. Implant 58 is thus supported from below by the part of main body 52a that is not removed to form notch 52c. The dimensions of implant 58 allow it to be inserted into bore 52b, but prevent it from falling out upon exiting bore 52b, i.e., upon exposure to or open communication with notch 52c, i.e., implant 58 is supported from below by the longitudinally-extending part of notch 52c as perhaps best understood in connection with FIG. 7C. Implant 58 is inside the cavity drilled into the sacrum and ilium when said implant extends beyond leading end 52g of cylindrical main body 52a.

A guide wire is then inserted through superior incision point 26 at a superior/interior angle about forty-five degrees (45°) towards superior. The medial/lateral angle is the same as the oblique angle on the C-arm, which is approximately thirty-five degrees (35°).

The guide wire is then guided into the SI joint. A second incision is made when the guide wire is properly positioned. The steps that follow the first incision are then repeated, i.e., joint locator 50 is inserted over the guide wire, directional cannula 52 is paced over the joint locator, and so on.

The procedure is concluded by inserting a guide wire through inferior incision point 28 at a superior/interior angle about forty-five degrees (45°) towards inferior. The medial/lateral angle is the same as the oblique angle on the C-arm, which is approximately thirty-five degrees (35°).

The guide wire is then guided into the SI joint. A third incision is made when the guide wire is properly positioned. The steps that follow the first and second incisions are then repeated, i.e., joint locator 50 is inserted over the guide wire, directional cannula 52 is paced over the joint locator, and so on.

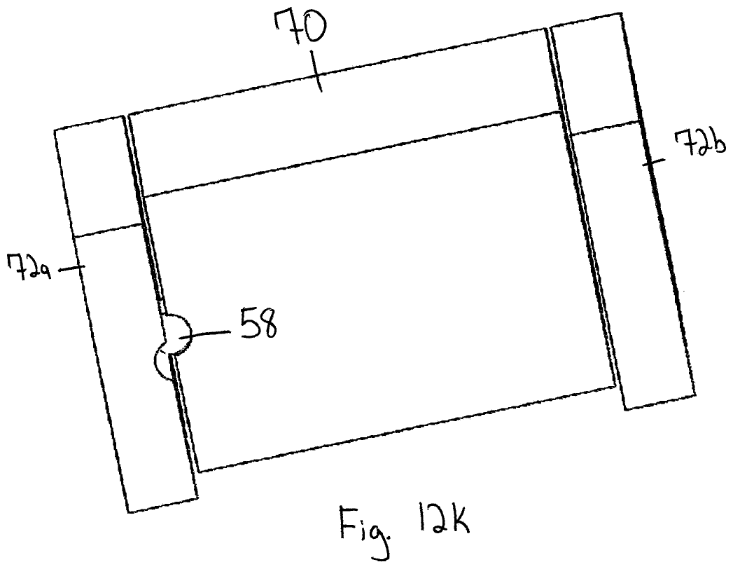

FIGS. 12A-K provide a simplified overview of the procedure. FIG. 12A is a front elevational diagrammatic representation of a sacrum 70 flanked by a pair of iliums 72a, 72b, showing two (2) sacroiliac joints 74a, 74b and FIG. 12B is a first perspective view of the diagrammatic representation of FIG. 12A, indicating how iliums 72a, 72b protrude forwardly relative to sacrum 70. FIG. 12C is a second perspective view of the diagrammatic representation of FIG. 12A and FIG. 12D is a view like FIG. 12C but adding notched cannula 52, showing how notch 52c accommodates a protruding ilium. FIG. 12E depicts the same parts as FIG. 12D but taken from a left perspective and FIG. 12F is the same view as FIG. 12E but including drill bit 56. FIG. 12G is the same as FIG. 12F but taken from a right perspective. FIG. 12H depicts cavity 76 after drilling is complete and the cannula and drill bit have been withdrawn. FIG. 12I is a like FIG. 12H but from a different perspective, FIG. 12J depicts implant 58 in drilled cavity 76, and FIG. 12K depicts the same structure as FIG. 12J but from a different perspective.

It will thus be seen that the objects set forth above, and those made apparent from the foregoing disclosure, are efficiently attained and since certain changes may be made in the above construction without departing from the scope of the invention, it is intended that all matters contained in the foregoing disclosure or shown in the accompanying drawings shall be interpreted as illustrative and not in a limiting sense.

It is also to be understood that the following claims are intended to cover all of the generic and specific features of the invention herein described, and all statements of the scope of the invention that, as a matter of language, might be said to fall therebetween.

1.-5. (canceled)

6. A method of positioning an implant into a sacroiliac joint, comprising the steps of:
providing said cannula with a cylindrical main body having a longitudinally-extending central bore formed therein, said central bore having a transverse profile that matches a transverse profile of said implant;
providing said cannula with a cut-out formed in its leading end;
providing a drill bit having a positive stop and inserting said drill bit into the cannula and drilling said drill bit in the sacral and ilium until said drill bit reaches said positive stop;
withdrawing said drill bit from said cannula;
inserting said implant into said central bore;
tamping said implant until said implant reaches said positive stop so that said implant is positioned inside the cavity drilled into the sacrum and ilium by said drilling; and
withdrawing said cannula.

7. A method of positioning an implant into a sacroiliac joint, comprising the steps of:
inserting a first guide wire into said sacroiliac joint;
positioning a joint locator over said first guide wire in ensleeving relation to said first guide wire;
positioning a directional cannula over said joint locator in ensleeving relation to said joint locator;
providing said directional cannula with a cylindrical main body having a longitudinally-extending central bore formed therein, said central bore having a transverse profile that matches a transverse profile of said implant;
forming a cut-out in a leading end of said directional cannula;
forming a pair of parallel, transversely spaced apart prongs that extend from said leading end of said directional cannula;
inserting said prongs into the sacroiliac joint;
retracting said joint locator and said first guide wire from said lumen of said directional cannula, leaving said directional cannula with said prongs disposed in the sacroiliac joint;
providing a drill guide having an elongate main body of rectangular cross-section and having a pair of parallel, longitudinally extending bores formed in said main body in eccentric relation to a longitudinal axis of symmetry of said drill guide;
providing a drill guide head that is enlarged relative to said main body;
forming a step in said main body that reduces the transverse extent of said main body near a leading end thereof, said step exposing a drill bit-accommodating semi-circular groove in each side of the leading end of said drill guide, said leading end being the part of said main body that extends distal of said step;
inserting said drill guide into the central bore of said directional cannula to verify placement of said directional cannula into said sacroiliac joint, said central bore of said directional cannula having a profile that enables it to slidingly receive said drill guide in the center of the larger implant-receiving central bore;
providing a drill bit having a positive stop and inserting said drill bit into a first bore of said eccentric bores and advancing said drill bit towards the ilium until it abuts the ilium;
drilling a first cavity in said ilium until said drill bit reaches said positive stop;
withdrawing said drill bit from said first bore and inserting said inserting drill bit into said second bore and drilling a second cavity in the ilium until said drill bit reaches said positive stop;
retracting said drill guide from said central bore of said directional cannula;
rotating said drill guide one hundred eighty degrees (180°) about its longitudinal axis of symmetry;
inserting said drill bit a second time into said first bore of said eccentric bores and advancing said drill bit towards the sacrum until it abuts the sacrum;
drilling a first cavity in said sacrum until said drill bit reaches said positive stop;
withdrawing said drill bit from said first bore and inserting said drill bit into said second bore a second time and drilling a second cavity in the sacrum until said drill bit reaches said positive stop;
said first and second ilium cavities merging with said first and second sacrum cavities to form a single cavity adapted to receive said fusion implant therein;
withdrawing said drill guide from said central bore of said directional cannula;
inserting said implant into said central bore;
tamping said implant until said implant reaches said positive stop so that said implant is positioned inside the cavity drilled into the sacrum and ilium by said first, second, third and fourth drillings; and
withdrawing said directional cannula.

8. (canceled)
